(19) United States
(12) Patent Application Publication
(10) Pub. No.: US 2022/0062022 A1
(43) Pub. Date: Mar. 3, 2022

(54) Device for Compensating a Shoulder Elevation in a Passive Exoskeleton

(71) Applicant: Hilti Aktiengesellschaft, Schaan (LI)

(72) Inventor: Simon KAESMANN, Muenchen (DE)

(21) Appl. No.: 17/414,840

(22) PCT Filed: Nov. 27, 2019
(86) PCT No.: PCT/EP2019/082690

§ 371 Date: Jun. 16, 2021

(30) Foreign Application Priority Data
Dec. 17, 2018 (EP) 18212835.5

(51) Int. Cl. A61F 5/37; A61H 1/02; A61F 5/01

(52) U.S. Cl. CPC A61F 5/3753; A61H 1/0281; A61F 5/013; A61F 2005/0148; A61F 2005/0139; A61F 2005/0144; A61H 2201/1616

(57) ABSTRACT

An exoskeleton for supporting an arm of a user includes a torso attachment device for releasably connecting the exoskeleton to a torso of the user, a bracket which is connectable to the torso attachment device, a lifting rod which is connectable to the torso attachment device via the bracket and which is reversibly movable in a first direction and a second direction relative to the bracket, a cantilever for supporting the arm of the user which is releasably connectable to the arm of the user, and a ratchet mechanism. The ratchet mechanism connects a first end of the lifting rod to a first end of the cantilever where the ratchet mechanism allows rotation of the cantilever in a first direction of rotation and blocks rotation of the cantilever in a second direction of rotation.

### BACKGROUND AND SUMMARY OF THE INVENTION

The present invention relates to an exoskeleton for supporting at least one arm of a user.

Exoskeletons that are available on the market and are available for the passive and/or active support of the extremities of a user, in particular the arms, usually include a hip fastening, a first and second supporting strut, and a first and second arm support. The first and the second supporting strut are vertically fastened to the left-hand and right-hand side, respectively, of the hip fastening. At the upper end of the first supporting strut, the first arm support is fastened in a rotatable manner via a pivot bearing. Likewise, at the upper end of the second supporting strut, the second arm strut is fastened in a rotatable manner via a further pivot bearing. The arm supports can be fixed in different angular positions relative to the supporting strut, such that the arms of the user can be rested on the deployed arm supports. With the aid of springs, the arm supports can push the arms of the user upward and as a result make it easier to lift a significant load (i.e., one with a great weight).

As a result of the anatomy of the human shoulder, as is schematically shown in FIG. 1a, when the arms are lifted, the shoulder joint is elevated at the same time. In exoskeletons according to the prior art, the pivot bearings between an upper supporting strut and arm support remain in one position, however, and do not follow the elevation of the human shoulder joint. This can result in a troublesome relative movement between the exoskeleton and the arms. This has the result that either the arms are forced onto a path of motion that is not desired by the user or the attachment points between the exoskeleton and human body slip. Movement restrictions and low wearing comfort are the result. An exoskeleton of this kind according to the prior art is shown for example in the patents U.S. Pat. No. 9,205,017 BB and U.S. Pat. No. 9,737,374 BB.

The object of the present invention is to solve the abovementioned problem and in particular to provide an improved exoskeleton for supporting at least one arm of a user.

This object is achieved by an exoskeleton for supporting at least one arm of a user.

According to the invention, the exoskeleton contains a torso attachment device for releasably connecting the exoskeleton to a torso of a user, at least one bracket, which is connectable to the torso attachment device, a lifting rod, which is connectable to the torso attachment device by means of the at least one bracket and which is reversibly movable in a first and a second direction relative to the bracket, a cantilever for supporting the arm of a user, which is releasably connectable to the arm of a user, and a ratchet mechanism for connecting a first end of the lifting rod to a first end of the cantilever, with the result that it is possible to selectively set a repeated, sectoral rotary movement of the cantilever in a first direction of rotation and a blocking action for the cantilever in a second direction of rotation. As a result, an exoskeleton is provided, which allows a resting possibility for the arms of a user which is adapted to the elevation of the human shoulder joint when the arm is lifted. As a result of this ergonomically or anatomically correct resting possibility for the arms of a user, better support can be achieved for the user when holding heavy loads.

According to an advantageous embodiment of the present invention, a coupling rod having a first and a second end can, the coupling rod being rotatably connected respectively to the cantilever by the first end and to the lifting rod by the second end via a compensating mechanism. By way of the coupling rod, the movement of the cantilever relative to the lifting rod is guided and supported. An undesired lateral movement of the cantilever or twisting or torsion of the cantilever with respect to the lifting rod and/or bracket can be prevented as a result.

According to an advantageous embodiment of the present invention, the compensating mechanism can be configured in the form of a slot at the second end of the coupling rod and of a pivot bearing on the lifting rod, wherein the pivot bearing is positioned in the slot. As a result, the compensating mechanism can be implemented in a simple manner. Furthermore, as a result of the slot at the end of the coupling rod, the cantilever can be lowered (i.e., moved downward) without exerting a force on further components.

According to an advantageous embodiment of the present invention, the compensating mechanism can be configured in the form of a slotted guide between the second end of the coupling rod and the lifting rod. This ensures reliable guidance of the second end of the coupling rod.

According to an advantageous embodiment of the present invention, the compensating mechanism can be configured in the form of a sector gear and of a rack, wherein the sector gear is rotatably connected to the lifting rod such that the teeth of the sector gear mesh with the teeth of the rack. The sector gear can also be referred to as a partial gear. As a result, effective guidance for avoiding an undesired lateral movement of the cantilever or twisting or torsion of the cantilever with respect to the lifting rod and/or bracket can be achieved in a simple manner. With the aid of the slot at the end of the coupling rod, the cantilever can be lowered (i.e., moved downward) without exerting a force on the sector gear.

According to an advantageous embodiment of the present invention, rather than the compensating mechanism, the articulated connection between the cantilever and the lifting rod can be embodied by a spindle or an actuator in order to move the cantilever upward. Rather than a mechanical coupling between the cantilever and the lifting rod, the angular position or the angle of the arm strut with respect to the lifting rod can also be determined via sensors, in order to correctly determine the elevation of the cantilever.

According to an advantageous embodiment of the present invention, the lifting rod and the rack can be configured as a piston-cylinder unit. As a result, the attachment of the lifting rod and the rack to the bracket can be implemented in a space-saving manner.

Further advantages will become apparent from the following description of the figures. The figures illustrate various exemplary embodiments of the present invention. The figures, the description and the claims contain numerous features in combination. A person skilled in the art will expediently also consider the features individually and combine them to produce useful further combinations.

In the figures, identical and similar components are denoted by the same reference signs.

### BRIEF DESCRIPTION OF THE DRAWINGS

FIG. 1a shows a front view of a human torso with a number of schematically illustrated joint points;

FIG. 1b shows a further front view of a human torso with a number of schematically illustrated joint points;

FIG. 2 shows a schematic side view of the device according to the invention for an exoskeleton for supporting an arm of a user in a first and a second position;

FIG. 3 shows a schematic illustration of the device according to the invention connected to an exoskeleton for supporting two arms of the user;

FIG. 4 shows a perspective view of the device according to the invention for an exoskeleton in the first setting;

FIG. 5 shows a side view of the device according to the invention for an exoskeleton in the first setting;

FIG. 6 shows a perspective view of the device according to the invention for an exoskeleton in the second setting;

FIG. 7 shows a side view of the device according to the invention for an exoskeleton in the second setting;

FIG. 8 shows a perspective view of the device according to the invention for an exoskeleton in a third setting; and

FIG. 9 shows a side view of the device according to the invention for an exoskeleton in the third setting.

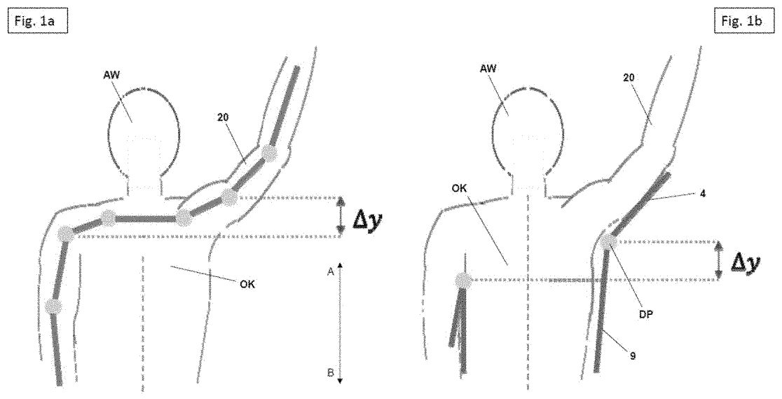

### DETAILED DESCRIPTION OF THE DRAWINGS

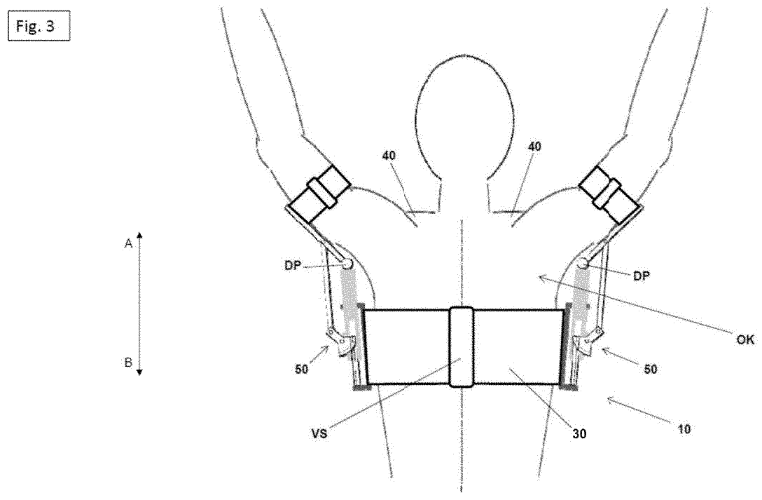

FIG. 3 shows a front view of a human upper body OK of a user AW, on which an exoskeleton 10 for supporting or increasing the work performance of the arms 20 of a user AW is arranged.

The exoskeleton 10 contains substantially a torso attachment device 30, a bracket 9, a lifting rod 7, a cantilever 4, and a ratchet mechanism 1.

The torso attachment device 30 serves to releasably connect the exoskeleton 10 to a torso (i.e., the upper body OK) of a user AW (cf. FIG. 3). The torso attachment device 30 can in this case be configured in the form of a hard shell, of a flexible chest strap or belt. Furthermore, the torso attachment device 30 contains a closure VS, arranged in the middle of the chest of the user AW, with which the torso attachment device 30, configured for example as a chest strap, can be opened and closed. The closure VS can be configured for example as a touch-and-close fastener or as a belt buckle.

According to an alternative embodiment (not shown in the figures) of the exoskeleton 10 according to the invention, the torso attachment device 30 can also be configured in the form of a hip belt and consequently be positioned on the hips or pelvis of the user AW. In addition, one or two straps can be fastened for fixing the torso attachment device 30 at the shoulders 40 of the user AW or for preventing the torso attachment device 30 from slipping in the arrow direction B. These straps (not illustrated in the figures) can be guided from the front side of the torso attachment device 30 configured as a chest strap or belt, over the shoulders 40 of the user AW (i.e., in a similar manner to suspenders), to the rear side of the torso attachment device 30.

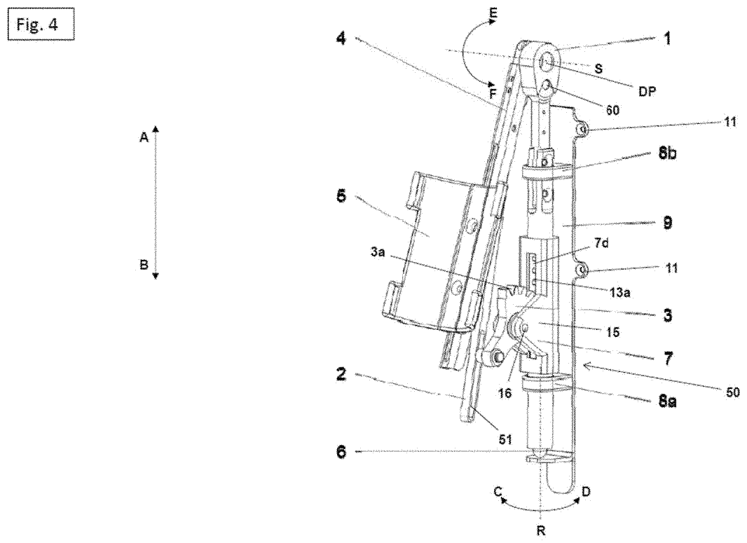

As shown in FIGS. 4 to 9, the bracket 9 is configured substantially as a flat metal sheet with four circular cutouts 11 and a first plain bearing 8a and a second plain bearing 8b. The four cutouts 11 can also be referred to as screw bosses and serve for laterally attaching the bracket 9 to the torso attachment device 30. For this purpose, four screws or rivets can be driven through the four cutouts 11 in the bracket 9 for fastening to the torso attachment device 30. Furthermore, the first and the second plain bearing 8a, 8b of the bracket 9 serve to receive and guide the lifting rod 7 at the bracket 9.

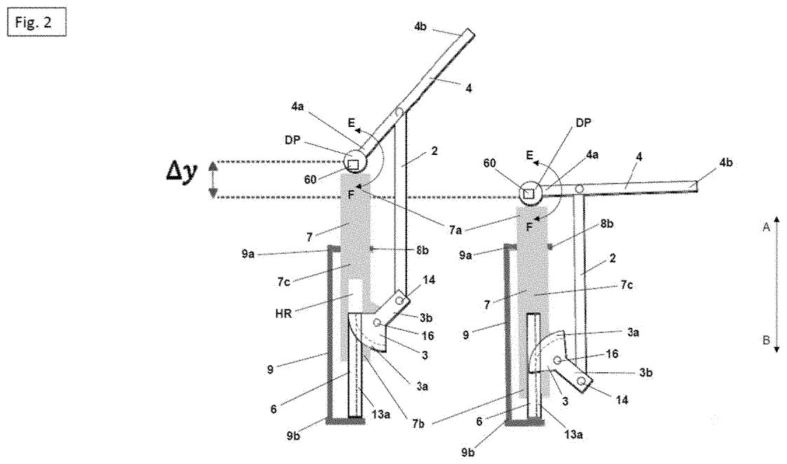

In FIG. 2, only the first plain bearing 8a is shown.

Each of the plain bearings 8a, 8b contains a circular opening. In this case, the first plain bearing 8a is positioned in the vicinity of a first end 9a of the bracket 9 and the second plain bearing 8b is positioned in the vicinity of a second end 9b of the bracket 9 such that the lifting rod 7 is guided through the respective circular openings of the plain bearings 8a, 8b and as a result is connected to the bracket 9 in a movable manner and in a reversible manner in the arrow directions A and B. Furthermore, the bracket 9 contains a support plate 12 at the second end 9b. As shown in FIG. 4, the lifting rod 7 is mounted in a rotatable manner on the support plate 12 when the lifting rod 7 is positioned on the bracket 9. Alternatively, the support plate 12 can also have a concave or curved shape directed toward the lifting rod 7.

According to an advantageous embodiment, the lifting rod 7, as shown in FIGS. 4 to 8, is configured substantially as a tube with a first end 7a and a second end 7b. This configuration of the lifting rod 7 can also be referred to as a pipe or sleeve. As is apparent in particular from FIG. 4, the first end 7a of the lifting rod 7 extends beyond the first end 9a of the bracket 9 in the arrow direction A. It is apparent from FIG. 2 that the lifting rod 7 configured as a tube contains a cavity HR, which extends from the second end 7b approximately as far as the middle 7c of the lifting rod 7. The cavity HR can also be referred to as a blind hole or cutout.

According to an advantageous embodiment, a support element 6 can be introduced into the cavity HR of the lifting rod 7. The support element 6 can be configured in a cylindrical manner and be adapted in its shape to the cavity HR of the lifting rod 7. The lifting rod 7 and the support element 6 are reversibly movable with respect to one another in the arrow directions A and B. The support element 6 serves substantially to support the lifting rod 7 and to increase stability when the lifting rod 7 is moved relative to the bracket 9. As will be described below, the support element 6 can also be configured with teeth and thus in the form of a rack.

As already mentioned above, the lifting rod 7 is connected to the bracket 9 such that the lifting rod 7 extends through the openings of the first and second plain bearings 8a, 8b. As a result, the lifting rod 7 is movable relative to the bracket 9 in a reversible manner in a first arrow direction A and a second arrow direction B. In other words: the lifting rod 7 can be moved in the arrow direction A and in the arrow direction B relative to the bracket 9. Moreover, the lifting rod 7 can be rotated about the axis of rotation R in the direction of rotation C or D relative to the bracket 9 by means of the first and second plain bearings 8a, 8b.

The cantilever 4 is configured substantially as a rod with a first end 4a and a second end 4b. The rod can also be referred to as an arm, support, bar or lever. As illustrated in FIGS. 4 to 9, an armrest 5 is positioned on the cantilever 4. The armrest 5 serves to receive and hold an upper arm of a user AW. In order to temporarily fix the arm to the armrest 5, an arm strap having a closure is provided. The arm strap and the closure are illustrated in FIG. 3. The closure is in this case configured for example as a touch-and-close fastener.

The ratchet mechanism 1 is configured substantially as a pivot bearing having a blocking action in a predetermined direction of rotation. Furthermore, the ratchet mechanism 1 contains a release device 60, with which the blocking action in the predetermined direction of rotation F is reversed again such that the ratchet mechanism 1 is free to rotate in the directions of rotation E and F. The release device 60 is indicated in FIG. 10 and is described in more detail below. The ratchet mechanism 1 can also be referred to as a ratchet or fine tooth ratchet. The ratchet mechanism 1 serves to connect the first end 7a of the lifting rod 7 to a first end 4a of the cantilever 4, with the result that it is possible to selectively set a repeated, sectoral rotary movement of the cantilever 4 about the axis of rotation S in a first direction of rotation E and a blocking action for the cantilever 4 in a second direction of rotation F. In other words: The cantilever 4 can be pulled freely in the direction of rotation E, for example by the arm of the user AW, which is connected to the cantilever 4 by means of the arm strap, and be blocked in the direction of rotation F with the aid of the blocking action of the ratchet mechanism 1. Since the ratchet mechanism 1 allows a free rotary movement of the cantilever 4 in the direction of rotation E about the pivot point, a user AW can move their arm upward in the arrow direction A in order for example to hold a power tool at a particular height. The power tool is not illustrated in the figures. With the aid of the blocking action of the ratchet mechanism 1 in the direction of rotation F, the cantilever 4 can be kept at the height intended by the user AW in order to provide a resting possibility for the arm by way of the cantilever blocked in the direction of rotation F. On account of the resting possibility provided by the cantilever 4 at the height intended by the user AW, holding of a heavy power tool can be substantially improved. As a result, the weight force of the power tool is dissipated via the arms to the cantilever 4, the lifting rod 7, the bracket 9 and finally to the torso attachment device 30.

The ratchet mechanism 1 is realized by a combination of two stop pawls, which transmit a pivoting movement of a lever only in one direction, while a movement in the opposite direction is automatically blocked. The operating principle is similar to that of freewheeling (for instance on a bicycle). In order that the release device 60 can reverse the blocking action of the ratchet mechanism 1 in a particular direction of rotation (in this case the direction of rotation F) again, it is possible for the release device 60 to separate the two stop pawls from one another such that they no longer act on one another in a blocking manner.

In addition to the embodiment with two stop pawls, one with a spring-loaded latching rocker is also possible. The ratchet mechanism 1 can also be realized by a push-through ratchet with a continuous square hole. In this case only a single (stop) pawl is necessary, wherein the effective direction of rotation is achieved by pushing through a square stub on one side of the ratchet or the other.

According to a further advantageous configuration, the exoskeleton 10 furthermore contains a coupling rod 2, which is configured substantially as a bar with a first end and a second end (cf. FIGS. 4 to 9). The coupling rod 2 is connected to the cantilever 4 in a rotatable manner by the first end and connected to the lifting rod 7 again in a rotatable manner by the second end via a compensating mechanism 50. The coupling rod 2 serves substantially as a support and guide when the cantilever 4 moves relative to the lifting rod 7.

According to an advantageous embodiment (not shown in the figures) of the exoskeleton 10 according to the invention, the compensating mechanism 50 can be configured in the form of a slot 51 at the second end of the coupling rod 2 and of a pivot bearing on the lifting rod 7. The pivot bearing is in this case positioned in the slot 51 and movable along the slot 51 and in a manner guided by the slot 51. With the aid of the compensating mechanism 50 configured as a slot and pivot bearing, a movement of the coupling rod 2, when the cantilever 4 is rotated relative to the lifting rod 7, can be compensated.

According to a further embodiment, shown in FIGS. 4 to 9, of the exoskeleton 10 according to the invention, the compensating mechanism 50 can be configured in the form of a slot 51 at the second end of the coupling rod 2, of a sector gear 3, of a rack 13, and of a pivot bearing 14.

The optional support element 6 is configured in the form of the rack 13 according to the embodiment, shown in FIGS. 4 to 9, of the exoskeleton 10 according to the invention. According to an advantageous embodiment of the exoskeleton 10 according to the invention, it is also possible for both a support element 6 and a rack 13 to be positioned alongside one another on the support plate 12.

The cylindrically configured rack 13 projects into the interior of the lifting rod 7 such that the longitudinal extent of the rack 13 and the longitudinal extent of the lifting rod 7 are oriented parallel to one another.

As is apparent in particular from FIG. 4, the lifting rod 7 has a cutout 7d, which provides access to the teeth of the rack 13. The rack 13 is connected to the second end 7b of the lifting rod 7 and in particular to the support plate 12 at the second end 7b of the lifting rod 7 so as to be rotatable about the axis of rotation R in the direction of rotation C or D by means of a pivot bearing (not shown). In this case, the rack 13 is connected to the support plate 12 such that the rack 13 cannot be moved in the arrow direction A or B bracket. As a result, there is no relative movement between the rack 13 and the bracket 9.

On account of the possibility of the rack 13 and the lifting rod 7 being able to move relative to one another in the arrow direction A or B and because the rack 13 is introduced into the interior of the lifting rod 7, the combination or the cooperation of the rack 13 and the lifting rod 7 can be referred to as a piston-cylinder unit. In this case, the rack 13 is implemented as a piston and the lifting rod 7 and in particular the cavity HR of the lifting rod 7, is implemented as a cylinder for receiving the piston configured as a rack.

Furthermore, the lifting rod 7 contains a holding device 15 having a pivot pin 16, which serves to receive and hold the sector gear 3. By way of the holding device 15 having the pivot pin 16, the sector gear 3 can be rotated relative to the lifting rod 7 such that the teeth 3a of the sector gear 3 can mesh with the teeth 13 of the rack 13. The sector gear 3 furthermore contains a lever element 3b, at the free end of which the pivot bearing 14 is positioned. The pivot bearing 14 is positioned in the slot 51 and freely movable along the slot 51 and guided along the slot 51.

The cantilever 4 can, as a result of its rotary mounting at the pivot point DP with respect to the bracket 9, be brought reversibly into a first, second or third position.

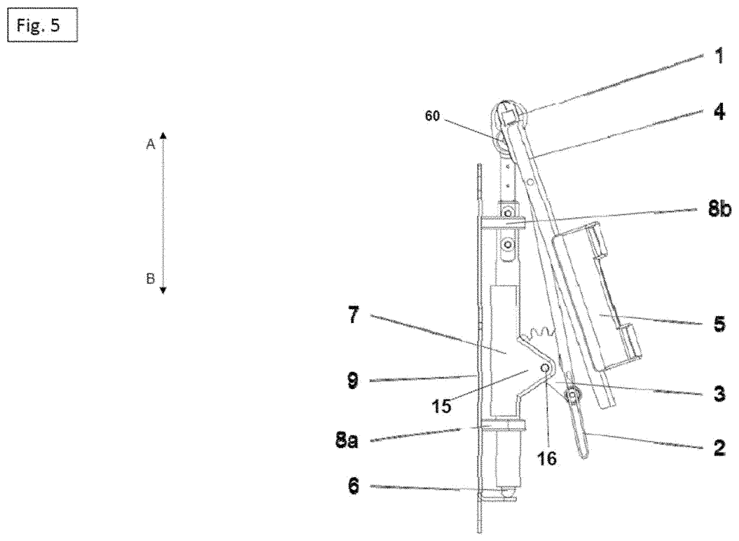

FIGS. 4 and 5 show the exoskeleton 10 with the cantilever 4 in the first position.

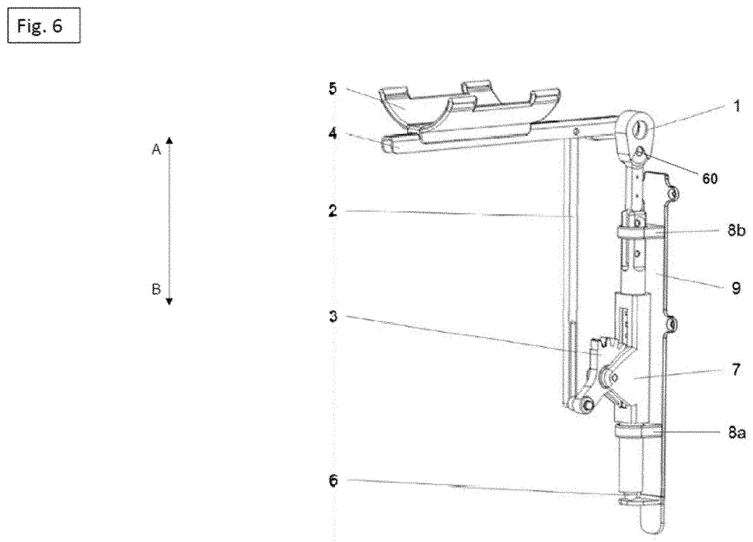

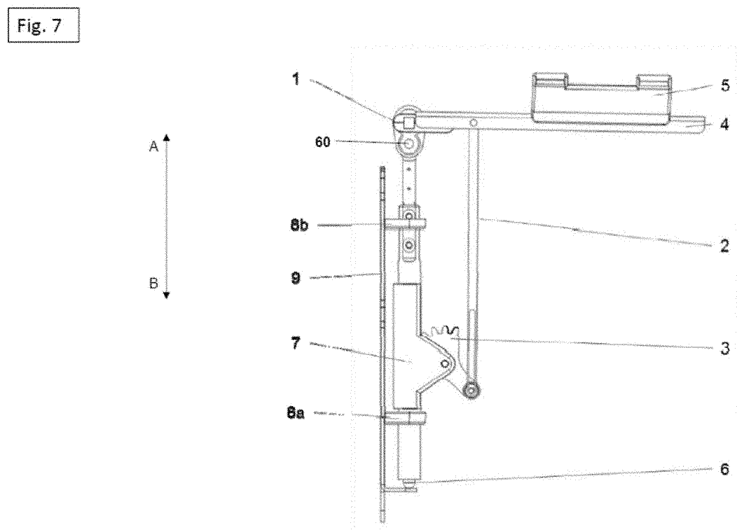

FIGS. 6 and 7 show the cantilever 4 in the second position.

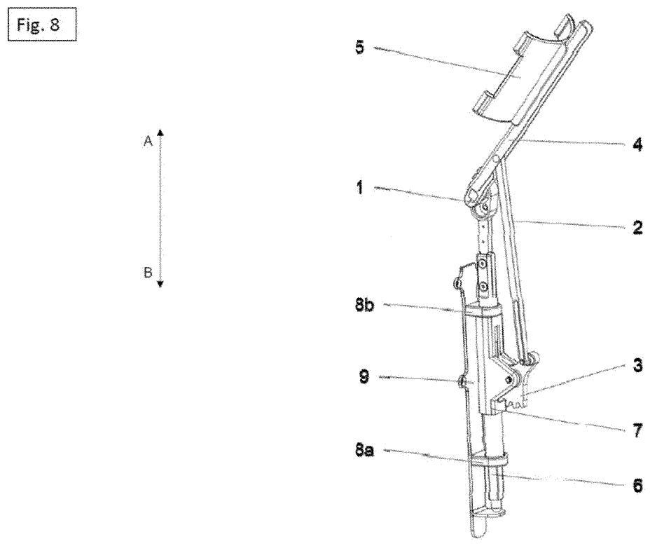

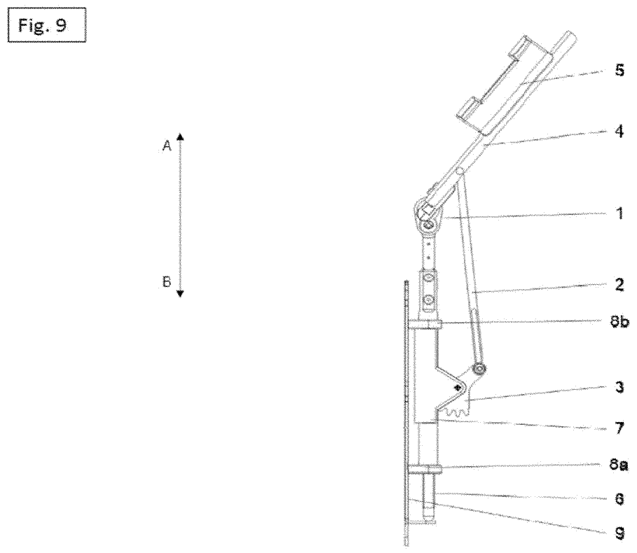

FIGS. 8 and 9 illustrate the cantilever 4 in the third position.

In the first position, the second end 4b of the cantilever 4 is inclined downward in the arrow direction B.

In the second position, the second end 4b of the cantilever 4 is substantially horizontal.

In the third position, the first end 4a of the cantilever 4 points upward in the arrow direction A.

When the user AW moves the arm that is connected to the cantilever 4 upward in the arrow direction A, the lifting rod 7 is likewise pulled upward in the direction A by means of the ratchet mechanism 1. Since the lifting rod 7 is mounted so as to be movable in the arrow direction A or B with respect to the bracket 9, the pivot point DP can compensate the travel Δy for compensating the anatomical movement of the human shoulder 40 during the lifting of an arm.

### LIST OF REFERENCE CHARACTERS

1: Ratchet mechanism

2: Coupling rod

3: Sector gear

3a: Teeth of the sector gear

3b: Lever element

4: Cantilever

4a: First end of the cantilever

4b: Second end of the cantilever

5: Armrest

6: Support element

7: Lifting rod

7a: First end of the lifting rod

7b: Second end of the lifting rod

7c: Middle of the lifting rod

7d: Cutout in the lifting rod

8a: First plain bearing

8b: Second plain bearing

9: Bracket

9a: First end of the bracket

9b: Second end of the bracket

10: Exoskeleton

11: Cutouts in the bracket

12: Support plate

13: Rack

13a: Teeth of the rack

14: Pivot bearing

15: Holding device

16: Pivot pin of the holding device

20: Arm of the user

30: Torso attachment device

40: Shoulders of the user

50: Compensating mechanism

51: Slot

60: Release device

HR: Cavity of the lifting rod

DP: Pivot point

OK: Upper body

AW: User

VS: Closure

R: Axis of rotation of the lifting rod

C, D: Directions of rotation of the lifting rod

S: Axis of rotation of the cantilever

E, F: Directions of rotation of the cantilever

1.-6. (canceled)

7. An exoskeleton for supporting an arm of a user, comprising:
a torso attachment device for releasably connecting the exoskeleton to a torso of the user;
a bracket which is connectable to the torso attachment device;
a lifting rod which is connectable to the torso attachment device via the bracket and which is reversibly movable in a first direction and a second direction relative to the bracket;
a cantilever for supporting the arm of the user which is releasably connectable to the arm of the user; and
a ratchet mechanism, wherein the ratchet mechanism connects a first end of the lifting rod to a first end of the cantilever and wherein the ratchet mechanism allows rotation of the cantilever in a first direction of rotation and blocks rotation of the cantilever in a second direction of rotation.

8. The exoskeleton according to claim 7 further comprising a coupling rod having a first end and a second end, wherein the coupling rod is rotatably connected to the cantilever by the first end and to the lifting rod via a compensating mechanism by the second end.

9. The exoskeleton according to claim 8, wherein the compensating mechanism comprises a slot at the second end of the coupling rod and a pivot bearing on the lifting rod and wherein the pivot bearing is disposed in the slot.

10. The exoskeleton according to claim 8, wherein the compensating mechanism comprises a slotted guide disposed between the second end of the coupling rod and the lifting rod.

11. The exoskeleton according to claim 8, wherein the compensating mechanism comprises a sector gear and a rack and wherein the sector gear is rotatably connected to the lifting rod such that teeth of the sector gear mesh with teeth of the rack.

12. The exoskeleton according to claim 11, wherein the lifting rod and the rack are configured as a piston-cylinder unit.
